(19) United States
(12) Patent Application Publication
(10) Pub. No.: US 2014/0074192 A1
(43) Pub. Date: Mar. 13, 2014

(54) BIOSTIMULATIVE ILLUMINATION APPARATUS

(71) Applicant: KAI-SHU SUNG, Taichung City (TW)

(72) Inventor: KAI-SHU SUNG, Taichung City (TW)

(21) Appl. No.: 14/070,394

(22) Filed: Nov. 1, 2013

Related U.S. Application Data
(63) Continuation-in-part of application No. 13/607,000, filed on Sep. 7, 2012.

(51) Int. Cl. A61N 5/06

(52) U.S. Cl. CPC A61N 5/0622
USPC 607/88

(57) ABSTRACT

A biostimulative illumination apparatus for treating patient tissues includes at least one light emitting diode which can generate at least one narrow-pulse focused wave band suitable to be used as low-power and non-parallel focused light beams for biostimulative illumination. The wave length of the focused light beam is from 600 nm to 850 nm, the energy density of the focused light beams is from 2 Joule/cm' to 16 Joule/cm' and the divergence angle of the light beams is between 1° to 7° that includes 1°, 2°, 3°, 4°, 5°, 6° and 7°.

### RELATED APPLICATIONS

This application is a continuation-in-part of U.S. patent application Ser. No. 13/607,000, filed on Sep. 7, 2012, which in turns is a continuation-in-part of U.S. patent application Ser. No. 13/087,310, filed on Apr. 14, 2011, which in turns is a continuation-in-part of U.S. patent application Ser. No. 11/925,638, filed on Oct. 26, 2007.

### BACKGROUND

1. Field of the Invention

The invention relates to a biostimulative illumination apparatus for treating patient tissues, and relates to a biostimulative illumination treatment method.

2. Description of the Related Art

Biostimulative illumination with low-power light is known as a treatment method. In conventional biostimulative illumination therapy, it always uses a focused light beam with a single frequency and a single energy, which limits the effect of the biostimulative illumination. Therefore, only using a focused light beam with a single frequency and a single energy, the biostimulative illumination therapy cannot be fully used. Additional, the conventional light beam has a big divergence angle, which makes the energy density of the focused light beam is small and resulting in that the biostimulative illumination effect is not good.

McDaniel's U.S. Pat. No. 6,663,659 discloses a method and apparatus for the photomodulation of living cells, in which the light source of the photomodulating apparatus is a narrowband multichromatic source emitting light within +/−20 nm of a dominant emissive wavelength, and a maximum light intensity of light emitted from the multichromatic source is no greater than about 4 J/cm².

U.S. Pat. No. 5, 259,380 to Mendes et al. discloses a light therapy system utilizes an array of light emitting diodes which emit noncoherent light in a narrow bandwidth centered at a designated wave length. The light emitting diodes are preferably selected with an angle of beam divergence of 12 degrees and a narrower angle of divergence, e.g. 8 degrees, will provide more concentrated light power at the focal point. However, the purpose of limiting the angle of the beam is not to provide most effective photomodulation to living cells.

To solve the above problems, the inventor of the invention researches many biostimulative illumination apparatuses and methods and successfully designs a biostimulative illumination apparatus of the invention.

### BRIEF SUMMARY

One object of the invention is to provide a biostimulative illumination apparatus for treating patient tissues. The biostimulative illumination apparatus includes at least one light emitting (at least one wave band) diode, which can generate at least one narrow-pulse focused wave band suitable to be used as low-power and non-parallel focused light beams for biostimulative illumination. The wave length of the focused light beams is from 600 nm to 850 nm, the energy density of the focused light beams is from 2 Joule/cm² to 16 Joule/cm² and the divergence angle of the focused light beams is between 1° to 7° that includes 1°, 2°, 3°, 4°, 5°, 6° and 7°, and preferred from 4° to 7° provide most effective photomodulation to living cells.

Another object of the invention is to provide a biostimulative illumination system for treating patient tissues. The biostimulative illumination apparatus includes at least one group of light emitting diode apparatus, a driver and a power source. The group of light emitting diode apparatus includes at least one light emitting diode which can generate at least one narrow-pulse focused wave band suitable to be used as low-power and non-parallel focused light beams for biostimulative illumination, wherein the wave length of the focused light beams is from 600 nm to 850 nm, the energy density of the focused light beams is from 2 Joule/cm² to 16 Joule/cm² and the divergence angle of the light beams is between 1° to 7° that includes 1°, 2°, 3°, 4°, 5°, 6° and 7°. The driver includes a voltage control circuit and a MCU. The power source is used for supplying power for the biostimulative illumination apparatus.

The biostimulative illumination system further comprises a key apparatus connected to the CPU, for inputting the desired data of light energy and frequency.

The biostimulative illumination system further comprises an over-current protection circuit for protecting the biostimulative illumination system and patients.

The biostimulative illumination system further comprises a pulse adjusting circuit for receiving pulse signal from the MCU and generating voltages to the light emitting diode.

In addition, the invention provides a biostimulative illumination treatment method. The method includes the following steps:

(a) providing at least one said light emitting diode apparatus, which can emitting a narrowpulse wave band focusing on a spectrum area of red light or near-infrared light, wherein the wavelength of the narrow-pulse wave band is from 600 nm to 850 nm.

(b) driving at least one light emitting diode apparatus to emit non-continuous and non-parallel focused light beams having a divergence angle between 1° to 7° that includes 1°, 2°, 3°, 4°, 5°, 6° and 7°.

(c) illuminating to the patient tissue by non-parallel focused light beams, whose energy density is from 4 Joule/cm² to 16 Joule/cm².

The invention has the following advantages:

Because the divergence angle of the light beam is between 1° to 7° that includes 1°, 2°, 3°, 4°, 5°, 6° and 7°, and the energy density of the focused light beam is from 2 Joule/cm² to 16 Joule/cm² in the invention, the light beam has a good penetrability to tissues and can reduce the recovering time of the wound.

According to the present invention, there are unexpected results listed below.

(1) 1°-7° divergence angle of limiting light beam will provide more concentrated light power at the focal point, either energy density or light power than 8° divergence angle.

(2) From 7°-1° gradually narrowing the light beam divergence angle, the light character will present the “coherency phenomenon” which we called the coherent light (the photons are in the same phase in time and space over a long distance), and consistency to the living cell. The light limited by the cells is coherent light. Therefore, a “resonance” will be occurred between the light beam and the radiated bio-photos in cell. Even, influencing one cell by the coherent light will trigger much larger effect than expected, because this cell communicates with hundreds of others and they will certainly share the information. That explanation is based upon the well-known butterfly effect (Lorenz, 1961) and the real coherent bio-stimulation effect will occur from 4° degree on to 1° degree. It's never occurred on just “wider angle” of 8° degree light beam. It's inventive to the optimal workable ranges by routine experimentation.

(3) The emission light beam with a narrowing divergent angle provides more power to the treatment site from Mendes. It's just the “common describe” and the efficiency is inverse square to the distance between the LEDs and tissue, and inversely proportional to the divergent angle. It's “geometric” change and not the Logarithmic (log) change. When the divergence angle of light beam gradually decrease from 7°-1°. The unexpected results will be happened by “coherency light produced” combined with the light permeability passing through the tissue which is called optical window of skin. It's estimated by two characters of optics and calculated by “Gaussian beam”. “Monochromatic light” means well polarized light photons; “High brightness” means huge quantity of photons, these will just occurred near 1° degree focal beam, not occurred by 8° or 12° degree. These changes on the narrowing angles) (4°-1° are unproportionally log category. It is not mentioned both by McDaniel and Mendes.

(4) “Gaussian beam”, an optical model describe the light beam power density including the amplitude as well as intensity over the cross section. When the light beam divergent angle eliminated to 1° degree, the Gaussian beam diagram will be forming “bell shaped”. It's a nice photons distribution phenomenon “coherency phenomenon” and will not be occurred on those light beams which divergent angle over 4° degrees. Additional, the loss energy of coherent light is great less than those of non-coherent light while penetrating through the tissue to the target cell.

### BRIEF DESCRIPTION OF THE DRAWINGS

These and other features and advantages of the various embodiments disclosed herein will be better understood with respect to the following description and drawings, in which like numbers refer to like parts throughout.

FIG. 1 is a schematic diagram of a biostimulative illumination apparatus of the invention.

FIG. 2 is a detail circuit diagram of FIG. 1.

FIG. 3 is an energy curve diagram of the first mode of the biostimulative illumination apparatus.

FIG. 4 is an energy curve diagram of the second mode of the biostimulative illumination apparatus.

FIG. 5 is an energy curve diagram of the third mode of the biostimulative illumination apparatus.

FIG. 6 is an energy curve diagram of the fourth mode of the biostimulative illumination apparatus.

FIG. 7 is an energy curve diagram of the fifth mode of the biostimulative illumination apparatus.

FIG. 8 is an energy curve diagram of the sixth mode of the biostimulative illumination apparatus.

### DETAILED DESCRIPTION

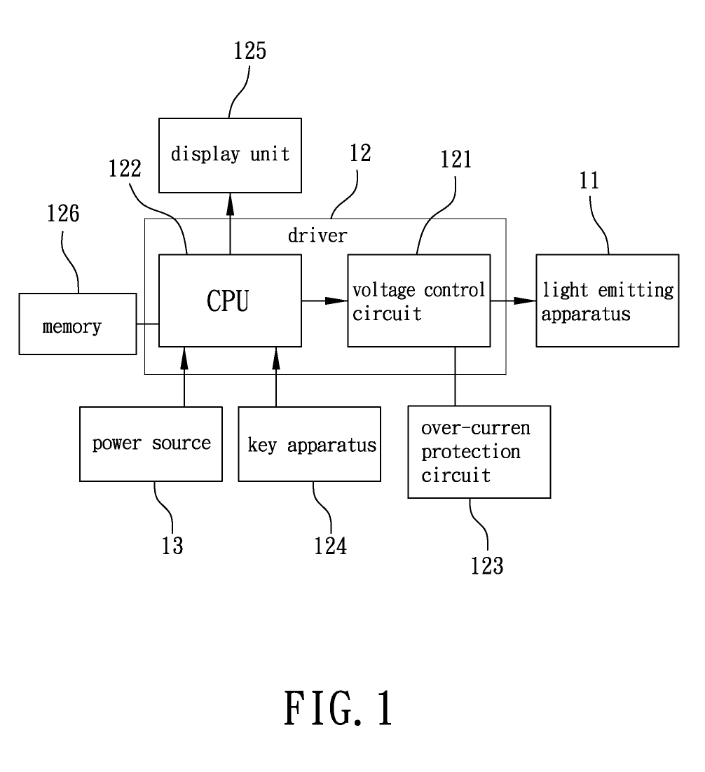

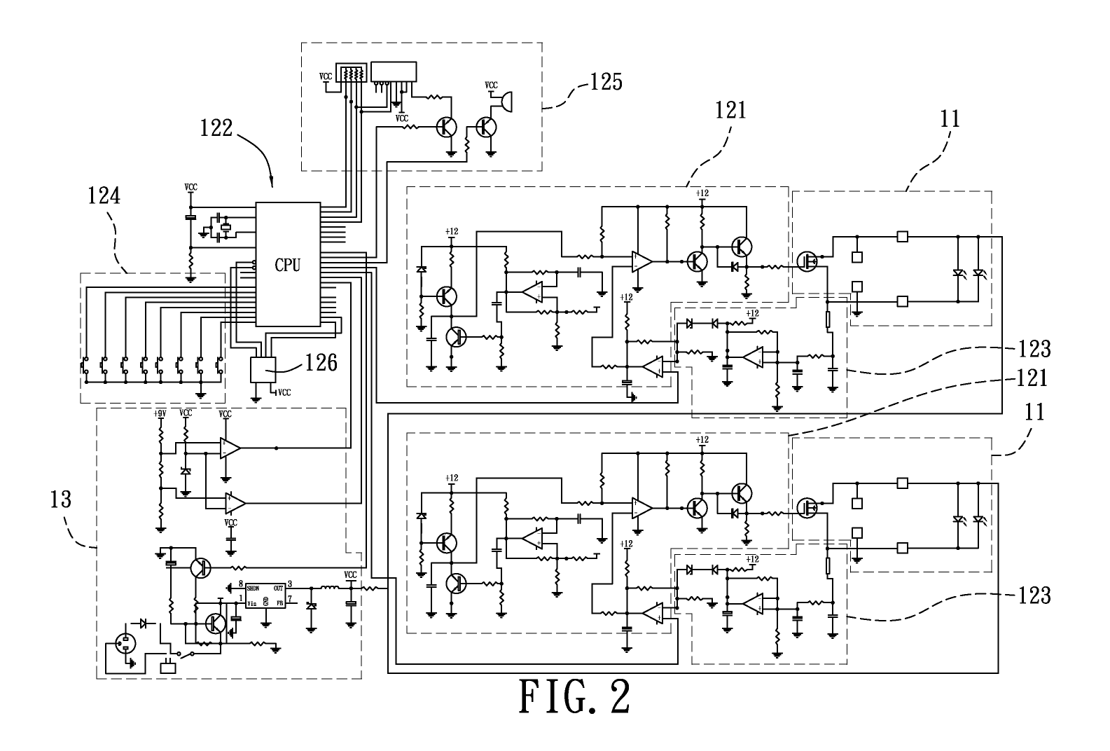

FIG. 1 is a schematic diagram of a biostimulative illumination apparatus of the invention. FIG. 2 is a detail circuit diagram of FIG. 1. The biostimulative illumination apparatus includes at least one group of light emitting diode apparatus 11, at least one driver 12 and a power source 13.

As shown in FIG. 1 and FIG. 2, the light emitting diode apparatus 11 includes at least one light emitting diode (LED), which can emit at least one narrow-pulse focused wave band such as a spectrum area of red light or near-infrared light. The narrow-pulse focused wave band is suitable to be used as low-power and non-parallel focused light beams for biostimulative illumination. The wave length of the focused light beams is preferred from 600 nm to 850 nm. The energy density of the light beam is from 2 Joule/cm² to 16 Joule/cm² and preferred from greater than 4 Joule/cm² and lower than 5 Joule/cm². Particularly, the divergence angle of the focused light beams provided by a specific LED is between 1° to 7° that includes 1°, 2°, 3°, 4°, 5°, 6° and 7°, and preferably from 4° to 7° in order to provide most effective photomodulation to living cells. The light emitting diode apparatus 11 can be consisted of light emitting diodes with different wavelengths and be in some different wave bands to support some different usages.

As shown in FIG. 2, the biostimulative illumination apparatus includes two groups of the light emitting diode apparatuses 11. The biostimulative illumination apparatus also can include one group or more groups of the light emitting diode apparatuses 11.

As shown in FIG. 2, the driver 12 drives the light emitting diode apparatus 11 to generate a focused light. It includes a voltage control circuit 121 and a Central processing unit (CPU) 122. The voltage control unit 121 receives modulated pulses from the CPU 122 and then generates different output voltages to the light emitting diode apparatus 11 for generating focused lights with different energy. In the embodiment shown in FIG. 2, the voltage control circuit 121 is a pulse adjusting circuit. The CPU 122 computes the frequency of the focused light beam and provides a corresponding control signal to the voltage control circuit 121.

As show in FIG. 2, the power source 13 can be a power supply or a battery. To get a better power supplying, the power source 13 can be managed by some power management methods.

As shown in FIG. 1 and FIG. 2, the biostimulative illumination apparatus further includes a memory 126 connected with the CPU 122. The memory 126 is utilized for storing messages such as a treatment method, which can be referred in a next treatment.

As show in FIG. 1 and FIG. 2, the biostimulative illumination apparatus further includes over-current protection circuit 123 for protecting the voltage control circuit 123. The biostimulative illumination apparatus further includes a key apparatus 124 connected to the CPU 122 for inputting the desired voltage and frequency, which will be displayed on a display unit 125.

The biostimulative illumination treatment method of using the biostimulative illumination apparatus of the invention includes the following steps:

(a) providing at least one said light emitting diode apparatus, which can emitting a narrow-pulse wave band focusing on a spectrum area of red light or near-infrared light, wherein the wavelength of the narrow-pulse wave band is from 600 nm to 850 nm.

(b) driving at least one light emitting diode apparatus to emit a non-continuous and non-parallel focused light beam having a divergence angle between 1° to 7° that includes 1°, 2°, 3°, 4°, 5°, 6° and 7° (preferred from 4° to 7° to provide most effective photomodulation to living cells).

(c) illuminating to the patient tissue by non-parallel focused light beams, whose energy density is from 2 Joule/cm² to 16 Joule/cm² and preferred greater than 4 Joule/cm² and lower than 5 Joule/cm².

The biostimulative illumination apparatus includes six operation modes for treatment operation.

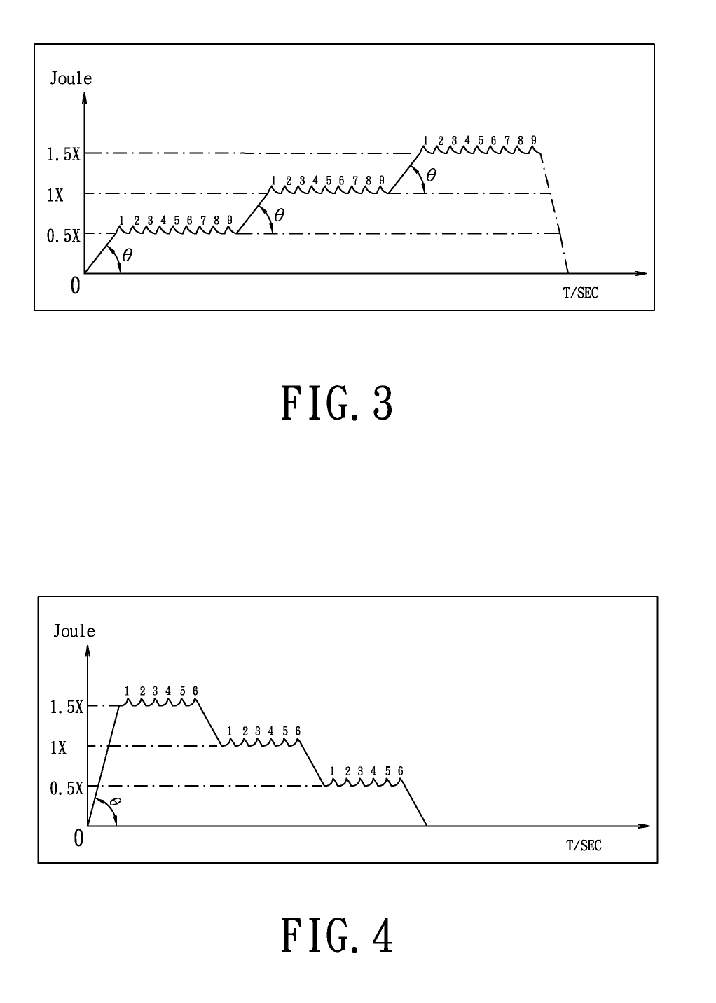

FIG. 3 is an energy curve diagram of the first mode of the biostimulative illumination apparatus. The treatment method of the first mode includes three illumination steps with three different energies. In the three illumination steps, the light energies are from 2 Joule/cm² to 16 Joule/cm² and increases by 0.5X Joule, 1X Joule and 2X Joule, wherein X is a variable, and can be set by a doctor. The illumination energy increases by an angle θ (θ≦20°) after each step. The steps can be repeated many times. In the first mode, the energy of illumination light is lightly fluctuated at 6 times or 9 times and each time is from 0.5 second to 1.5 seconds. The fluctuating amplitude is lower than 20% and the fluctuating way is quickly ascending and slowly descending.

FIG. 4 is an energy curve diagram of the second mode of the biostimulative illumination apparatus. The treatment method of the second mode includes three illumination steps with three different energies. In the three illumination steps, the light energies are from 2 Joule/cm² to 16 Joule/cm² and increases by 0.5X Joule, 1X Joule and 2X Joule, wherein X is a variable and set by a doctor, the illumination energy increases by an angle θ (θ≧45°) after each step. The steps can be repeated many times. In the second mode, the energy of illumination light is lightly fluctuated at 6 times or 9 times and each time is from 0.5 second to 1.5 seconds. The fluctuating amplitude is lower than 20% and the fluctuating way is slowly ascending and quickly descending.

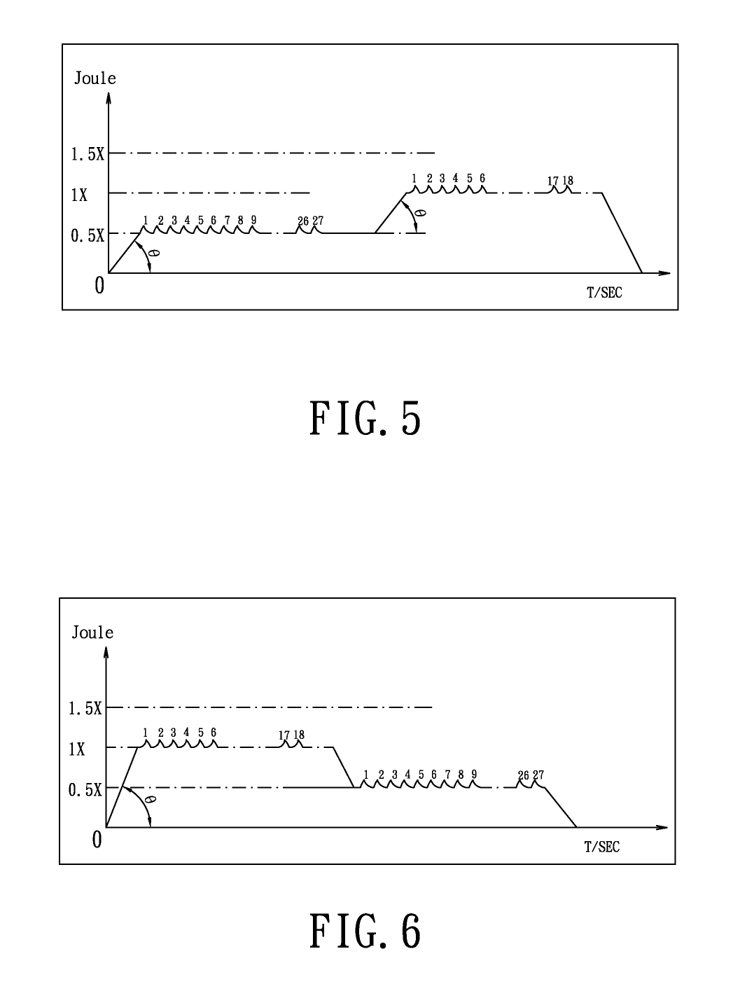

FIG. 5 is an energy curve diagram of the third mode of the biostimulative illumination apparatus. The treatment method of the third mode includes two illumination steps with different energies. In the two illumination steps, the light energies are from 2 Joule/cm² to 16 Joule/cm² and increases by 0.5X Joule, 1X Joule and 2X Joule, wherein X is a variable and set by a doctor. The illumination energy increases by an angle θ (θ≦60°) after each step. The steps can be repeated many times. In the third mode, the energy of illumination light lightly fluctuates at many times and each time is from 0.5 second to 1.5 second. The fluctuating amplitude is lower than 20%. In the first step, the light fluctuates 9 or 27 times and the fluctuating way is quickly ascending and slowly descending. In the second step, the light fluctuates 6 or 18 times and the fluctuating way is slowly ascending and quickly descending.

FIG. 6 is an energy curve diagram of the fourth mode of the biostimulative illumination apparatus. The treatment method of the fourth mode includes two illumination steps with different energies. In the two illumination steps, the light energies are from 2 Joule/cm² to 16 Joule/cm² and increases by 0.5X Joule, 1X Joule and 2X Joule, wherein X is a variable. As set by a doctor, the illumination energy increases by an angle θ (θ≦60°) by each step. The steps can be repeated many times. In the fourth mode, the energy of illumination light lightly fluctuates at many times and each time is from 0.5 second to 1.5 seconds. The fluctuating amplitude is lower than 20%. In the fourth step, the light fluctuates 6 or 18 times and the fluctuating way is quickly ascending and slowly descending. In the second step, the light fluctuates 9 or 27 times and the fluctuating way is quickly ascending and slowly descending.

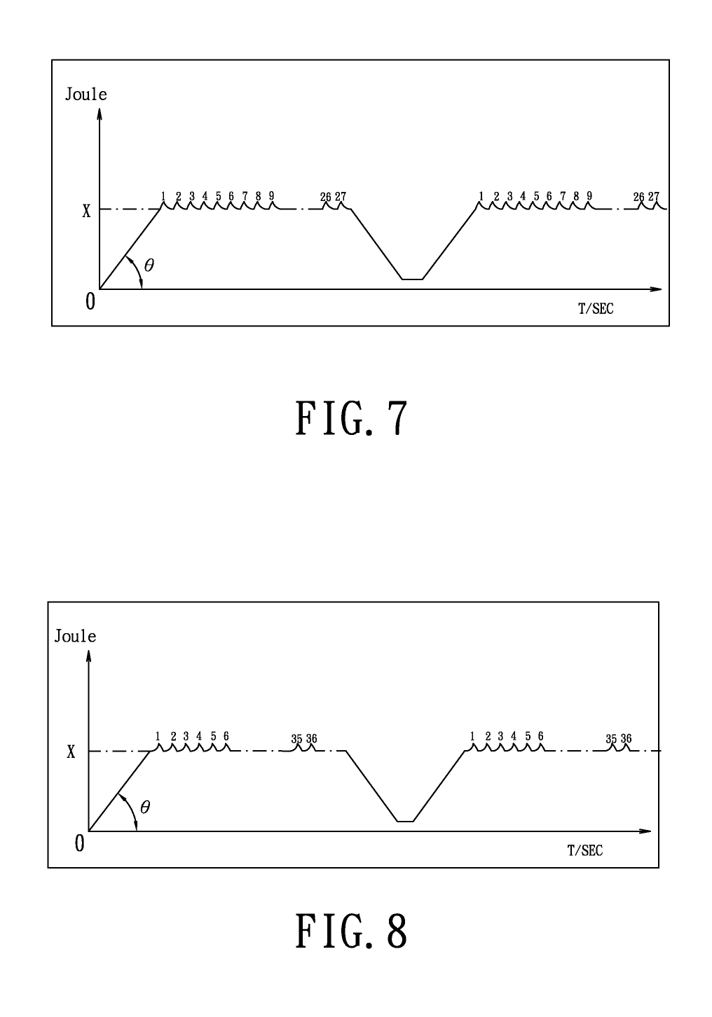

FIG. 7 is an energy curve diagram of the fifth mode of the biostimulative illumination apparatus. The treatment method of the fifth mode includes an illumination step with a kind of light illumination energy. In the illumination step, the light energy is X Joule, wherein X is a variable and set by a doctor. The illumination energy increases by an angle θ (about 45°). The step can be repeated many times. In the fifth mode, the energy of illumination light lightly fluctuates at 9 or 27 times and each time is between 0.5 to 1.5 second. The fluctuating amplitude is lower than 20%. The fluctuating way is quickly ascending and slowly descending.

FIG. 8 is an energy curve diagram of the sixth mode of the biostimulative illumination apparatus. The treatment method of the sixth mode includes an illumination step with a kind of light illumination energy. In the illumination step, the light energy is X Joule, wherein X is a variable and set by a doctor. The illumination energy increases by an angle θ (the θ is approximately equal to 45°). The step can be repeated many times. In the fifth mode, the energy of illumination light lightly fluctuates at 6 or 18 times and each time is from 0.25 second to 1.0 second. The fluctuating amplitude is lower than 20%. The fluctuating way is slowly ascending and quickly descending.

The following description is to explain the variable X. For example, in the first mode,

for x=4, the three light energies of the three steps are:
0.5X 4 Joule=2 Joule
1X 4 Joule=4 Joule
1.5X 4 Joule=6 Joule.

for x=5, the three light energies of the three steps are:
0.5X 5 Joule=2.5 Joule
1X 5 Joule=5 Joule
1.5X 5 Joule=7.5 Joule.

for x=10.66, the three light energies of the three steps are:
0.5X 10.66 Joule=5.33 Joule
1X 10.66 Joule=10.66 Joule
1.5X 10.66 Joule=15.99 Joule.

So when X is from 4 to 10.66, the light energy will not be out of the extent from 2 Joule/cm² to 16 Joule/cm². In the second to the sixth modes, the X follows the same principle as the first mode.

In operation, X and the illumination time can be set by a doctor, and be recorded in a memory.

If energy density of the illumination light is over 16 Joule/cm², the illuminated cells will react slowly and the biostimulative illumination effect will get a reverse result. If energy density of the illumination light is lower than 2 Joule/cm², the illuminated cells will have a worse reaction, even no reaction. Therefore, to get a better biostimulative illumination effect, the energy density of the illumination light of the invention is selected from 2 Joule/cm² to 16 Joule/cm² and preferred greater than 4 Joule/cm² and lower than 5 Joule/cm².

In addition, if the divergence angle is too big, the light energy is not enough and biostimulative illumination effect is not good. To get a better biostimulative illumination effect, the light divergence angle of the invention is between 1° to 7° that includes 1°, 2°, 3°, 4°, 5°, 6° and 7°, and preferred from 4° to 10° to provide most effective photomodulation to living cells.

The above description is given by way of example, and not limitation. Given the above disclosure body, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A biostimulative illumination apparatus for treating patient tissues, comprising at least one light emitting diode which can generate at least one narrow-pulse focused wave band suitable to be used as low-power and non-parallel focused light beams for biostimulative illumination, wherein the wave length of the focused light beams is from 600 nm to 850 nm, the energy density of the focused light beam is from 2 Joule/cm² to 16 Joule/cm² and the divergence angle of the focused light beams is between 1° to 7° that includes 1°, 2°, 3°, 4°, 5°, 6° and 7° to provide most effective photomodulation to living cells.

2. The biostimulative illumination apparatus of claim 1, wherein the divergence angle of the light beam is from 4° to 7° in order to provide effective photomodulation to living cells.

3. The biostimulative illumination apparatus of claim 1, wherein the energy density of the focused light beam is greater than 4 Joule/cm² and lower than 5 Joule/cm².
